(19) United States
(12) Patent Application Publication
(10) Pub. No.: US 2025/0387085 A1
(43) Pub. Date: Dec. 25, 2025

(54) IMAGING CONTROL DEVICE, MEDICAL IMAGE CAPTURING DEVICE, IMAGING PARAMETER SETTING METHOD, AND PROGRAM

(71) Applicant: FUJIFILM Corporation, Tokyo (JP)

(72) Inventor: Tomohiro GOTO, Tokyo (JP)

(73) Assignee: FUJIFILM Corporation, Tokyo (JP)

(21) Appl. No.: 19/242,653

(22) Filed: Jun. 18, 2025

(30) Foreign Application Priority Data
Jun. 21, 2024 (JP) 2024-100465

(51) Int. Cl. A61B 5/00; A61B 5/055; G01R 33/54; G01R 33/567; G06T 7/00

(52) U.S. Cl. CPC A61B 5/7285; A61B 5/055; G01R 33/543; G01R 33/5673; G06T 7/0012; G06T 2207/10088; G06T 2207/30004

(57) ABSTRACT

An imaging control device, a medical image capturing device, an imaging parameter setting method, and a program that implement adjustment of an imaging parameter suitable for a change in biological information are provided. An imaging control device acquires biological information of a subject, in a case where a second set value based on the biological information of the subject is changed from a first set value set in advance for a first imaging parameter for which the biological information of the subject is set, determines whether or not a second imaging parameter needs to be adjusted, and in a case where a determination that the second imaging parameter needs to be adjusted is made, displays parameter adjustment suggestion information related to adjustment of the second imaging parameter.

### CROSS-REFERENCE TO RELATED APPLICATION

The present application claims priority under 35 U.S.C § 119(a) to Japanese Patent Application No. 2024-100465 filed on Jun. 21, 2024, which is hereby expressly incorporated by reference, in its entirety, into the present application.

### BACKGROUND OF THE INVENTION

### 1. Field of the Invention

The present disclosure relates to an imaging control device, a medical image capturing device, an imaging parameter setting method, and a program.

### 2. Description of the Related Art

A medical image capturing device that performs imaging synchronized with a biological signal indicating biological information such as a heart rate and respiration has been known. For example, an MRI device monitors a heart rate during imaging of a heart and measures an MR signal while synchronizing with the heart rate. MRI is the abbreviation for Magnetic Resonance Imaging. MR is the abbreviation for Magnetic Resonance.

JP2012-147921A discloses an MRI device that supports setting of a synchronization parameter related to synchronized imaging during synchronized imaging synchronized with a biological signal. The device according to JP2012-147921A monitors a biological signal of a subject to be imaged and displays a waveform of the biological signal generated from the biological signal and a synchronization parameter set by an operator in a superimposed manner as an image.

WO2015/093296A discloses an MRI device that performs imaging of a subject in synchronization with biological information of the subject. The device according to WO2015/093296A calculates a prediction value of an SAR based on a period of the biological information and, in a case where the prediction value of the SAR exceeds a limit value, presents a plurality of suggestions to change an imaging condition such that the SAR does not exceed the limit. SAR is the abbreviation for Specific Absorption Rate which is an English term indicating an amount of absorption of a radio-frequency magnetic field pulse per unit time and unit mass.

### SUMMARY OF THE INVENTION

In the imaging synchronized with the biological information, an imaging parameter related to the biological information may have to be changed in a case where the biological information significantly changes. There are multiple imaging parameters related to the biological information. Changing multiple imaging parameters requires time and labor. In particular, manually changing the imaging parameters is repetitive and arduous.

JP2012-147921A does not provide disclosure and indication related to changing of the imaging parameter caused by a change in the biological information.

WO2015/093296A provides disclosure related to presentation of the suggestions to change the imaging parameter such that the SAR does not exceed the limit value in a case where the SAR exceeds the limit value, but does not provide disclosure and indication related to changing of the imaging parameter caused by a change in the biological information.

The present disclosure has been conceived in view of such circumstances, and an object of the present disclosure is to provide an imaging control device, a medical image capturing device, an imaging parameter setting method, and a program that implement preferable adjustment of an imaging parameter caused by a change in biological information.

According to a first aspect of the present disclosure, there is provided an imaging control device that sets an imaging parameter applied to imaging of a subject including a synchronized imaging task in which biological information of the subject is used, in a medical image capturing device which performs the imaging, the imaging control device comprising: a processor; and a memory storing a program to be executed by the processor, in which the processor is configured to acquire the biological information of the subject, in a case where a second set value based on the acquired biological information of the subject is changed from a first set value set in advance for a first imaging parameter for which the biological information of the subject is set, determine whether or not a second imaging parameter different from the first imaging parameter needs to be adjusted, and in a case where a determination that the second imaging parameter needs to be adjusted is made, display parameter adjustment suggestion information related to adjustment of the second imaging parameter.

According to the imaging control device according to the first aspect, the parameter adjustment suggestion information related to adjustment of the second imaging parameter that needs to be adjusted because of the change in the first imaging parameter from the first set value to the second set value is displayed. Accordingly, adjustment of the second imaging parameter linked to the change in the first imaging parameter is simplified, and operations of an operator and effects on a captured image that depends on a skill level of the operator are reduced. That is, time and labor of input of the operator are reduced, and occurrence of forgetting to input the second imaging parameter, input errors, and the like is suppressed.

According to a second aspect of the imaging control device, in the imaging control device of the first aspect, the processor may be configured to display a suggestion to adjust some of a plurality of the second imaging parameters as the parameter adjustment suggestion information.

According to a third aspect of the imaging control device, in the imaging control device of the second aspect, the processor may be configured to acquire selection information for selecting the displayed suggestion to adjust the second imaging parameter, and automatically adjust a third imaging parameter different from the selected second imaging parameter.

According to a fourth aspect of the imaging control device, in the imaging control device of the first aspect, the processor may be configured to display information indicating an effect on the imaging of the subject as the parameter adjustment suggestion information.

According to a fifth aspect of the imaging control device, in the imaging control device of any one of the first to fourth aspects, the processor may be configured to automatically acquire the second set value.

According to a sixth aspect of the imaging control device, in the imaging control device of any one of the first to fifth aspects, the processor may be configured to, in a case where a plurality of the synchronized imaging tasks are performed, acquire the biological information for each synchronized imaging task, and set a latest second set value for each synchronized imaging task.

According to a seventh aspect of the imaging control device, in the imaging control device of any one of the first to sixth aspects, the processor may be configured to calculate the second set value based on the biological information acquired in a period of two or more cycles of a repeating cycle of the biological information.

According to an eighth aspect of the imaging control device, in the imaging control device of any one of the first to seventh aspects, the processor may be configured to acquire a change in a biological information value that is a value of the biological information, and in a case where the acquired change in the biological information value exceeds a determined value, provide notification of abnormality information indicating abnormality in the biological information value.

According to a ninth aspect of the imaging control device, in the imaging control device of any one of the first to eighth aspects, the processor may be configured to, in a case where the determination that the second imaging parameter needs to be adjusted is made, display a plurality of pieces of the parameter adjustment suggestion information.

According to a tenth aspect of the imaging control device, in the imaging control device of any one of the first to ninth aspects, the processor may be configured to determine whether or not the second set value based on the acquired biological information of the subject is changed from the first set value set in advance for the first imaging parameter for which the biological information of the subject is set.

According to an eleventh aspect of the imaging control device, in the imaging control device of any one of the first to ninth aspects, the processor may be configured to, in a case where a change in the second set value from the first set value of the first imaging parameter is greater than or equal to a determined value, determine that the second imaging parameter needs to be adjusted.

According to a twelfth aspect of the present disclosure, there is provided a medical image capturing device that performs imaging of a subject including a synchronized imaging task in which biological information of the subject is used, the medical image capturing device comprising a processor, and a memory storing a program to be executed by the processor, in which the processor is configured to acquire the biological information of the subject, in a case where a second set value based on the acquired biological information of the subject is changed from a first set value set in advance for a first imaging parameter for which the biological information of the subject is set, determine whether or not a second imaging parameter different from the first imaging parameter needs to be adjusted, and in a case where a determination that the second imaging parameter needs to be adjusted is made, display parameter adjustment suggestion information related to adjustment of the second imaging parameter.

According to the medical image capturing device according to the twelfth aspect of the present disclosure, the same effects as the imaging control device according to the first aspect can be achieved. Configuration requirements of the imaging control device according to the second to eleventh aspects may be applied to configuration requirements of the medical image capturing device according to other aspects.

According to a thirteenth aspect of the present disclosure, there is provided an imaging parameter setting method performed by a computer functioning as a medical image capturing device that performs imaging of a subject including a synchronized imaging task in which biological information of the subject is used, the imaging parameter setting method comprising: acquiring the biological information of the subject; determining, in a case where a second set value based on the acquired biological information of the subject is changed from a first set value set in advance for a first imaging parameter for which the biological information of the subject is set, whether or not a second imaging parameter different from the first imaging parameter needs to be adjusted; and displaying, in a case where a determination that the second imaging parameter needs to be adjusted is made, parameter adjustment suggestion information related to adjustment of the second imaging parameter.

According to the imaging parameter setting method according to the thirteenth aspect of the present disclosure, the same effects as the imaging control device according to the first aspect can be achieved. The configuration requirements of the imaging control device according to the second to eleventh aspects may be applied to configuration requirements of the imaging parameter setting method according to other aspects.

According to a fourteenth aspect of the present disclosure, there is provided a program causing a computer functioning as a medical image capturing device that performs imaging of a subject including a synchronized imaging task in which biological information of the subject is used, to implement a function of acquiring the biological information of the subject, a function of determining, in a case where a second set value based on the acquired biological information of the subject is changed from a first set value set in advance for a first imaging parameter for which the biological information of the subject is set, whether or not a second imaging parameter different from the first imaging parameter needs to be adjusted, and a function of displaying, in a case where a determination that the second imaging parameter needs to be adjusted is made, parameter adjustment suggestion information related to adjustment of the second imaging parameter.

According to the program according to the fourteenth aspect of the present disclosure, the same effects as the imaging control device according to the first aspect can be achieved. The configuration requirements of the imaging control device according to the second to eleventh aspects may be applied to configuration requirements of the program according to other aspects.

According to the present disclosure, the parameter adjustment suggestion information related to adjustment of the second imaging parameter that needs to be adjusted because of the change in the first imaging parameter from the first set value to the second set value is displayed. Accordingly, adjustment of the second imaging parameter corresponding to the change in the set value of the first imaging parameter is implemented.

### BRIEF DESCRIPTION OF THE DRAWINGS

FIG. 1 is a perspective view illustrating an exterior of an MRI device.

FIG. 2 is a schematic diagram illustrating an internal configuration of the MRI device.

FIG. 3 is a flowchart illustrating a procedure of an imaging parameter setting method of a medical image according to an embodiment.

FIG. 4 is a schematic diagram illustrating a configuration example of a display screen.

FIG. 5 is a schematic diagram illustrating a display example of an imaging parameter setting region illustrated in FIG. 4.

FIG. 6 is a schematic diagram illustrating a first example of a parameter adjustment suggestion information display screen.

FIG. 7 is a schematic diagram illustrating a second example of the parameter adjustment suggestion information display screen.

FIG. 8 is a schematic diagram illustrating a third example of the parameter adjustment suggestion information display screen.

FIG. 9 is a timing chart illustrating an example of an imaging parameter in electrocardiogram-synchronized imaging.

FIG. 10 is a timing chart illustrating a case where Beat Rate is changed.

FIG. 11 is a schematic diagram illustrating a first change example of the imaging parameter.

FIG. 12 is a schematic diagram illustrating a second change example of the imaging parameter.

FIG. 13 is a schematic diagram illustrating another display example of the imaging parameter setting region.

FIG. 14 is a schematic diagram of heart rate detection in calculating a heart rate.

FIG. 15 is a flowchart illustrating a procedure of abnormality detection in biological information.

FIG. 16 is a schematic diagram illustrating a notification example of abnormality in the biological information.

### DESCRIPTION OF THE PREFERRED EMBODIMENTS

Hereinafter, a preferred embodiment of the present invention will be described in detail with reference to the accompanying drawings. In the following description and the accompanying drawings, the same constituents will be designated by the same reference numerals, and description thereof will not be repeated. In the following embodiment, a list of a plurality of constituents illustrated may be interpreted as including at least one of the plurality of constituents.

### Configuration Example of MRI Device

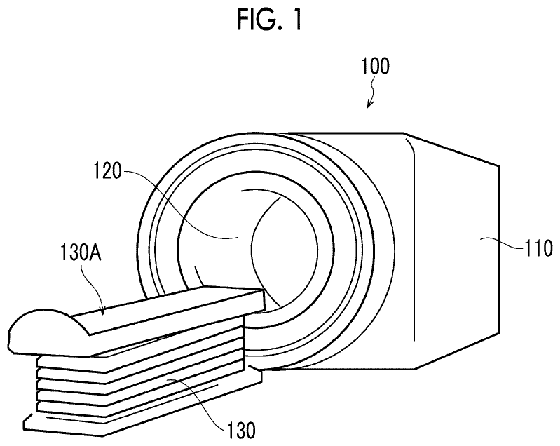

FIG. 1 is a perspective view illustrating an exterior of an MRI device. An MRI device 100 that is a magnetic resonance imaging device comprises a gantry 110 that is a device body, and a bed 130. The bed 130 comprises a top plate 130A and is disposed on a front side of a bore 120 that is a cylindrical imaging space provided in the gantry 110. The top plate 130A can be advanced into the bore 120 and retracted from the bore 120 using a top plate drive mechanism provided in the bed 130. The top plate drive mechanism is not illustrated.

The bed 130 may be configured to be fixed to the gantry 110 or may be a dockable bed that is a mobile bed attachable to and detachable from the gantry 110. The MRI device 100 is an example of a medical image capturing device of the present disclosure.

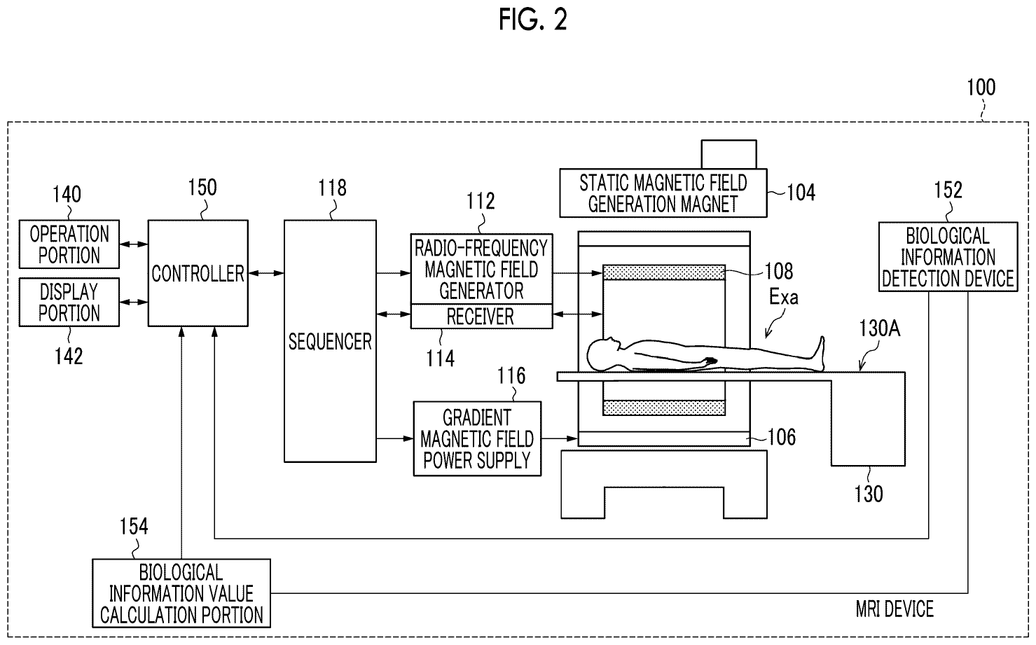

FIG. 2 is a schematic diagram illustrating an internal configuration of the MRI device. The MRI device 100 comprises a static magnetic field generation magnet 104, a gradient magnetic field coil 106, and an RF transmit coil 108. RF is the abbreviation for Radio Frequency.

The MRI device 100 comprises a radio-frequency magnetic field generator 112, a receiver 114, a gradient magnetic field power supply 116, and a sequencer 118. The MRI device 100 comprises an operation portion 140, a display portion 142, a controller 150, a biological information detection device 152, and a biological information value calculation portion 154.

A subject Exa is placed on the top plate 130A of the bed 130 and is disposed in the imaging space. That is, the top plate 130A on which the subject Exa is placed moves into the bore 120. Accordingly, a part of the subject Exa to be examined is disposed at the center of a static magnetic field in the bore 120.

The static magnetic field generation magnet 104 generates a uniform static magnetic field in the imaging space. The static magnetic field generation magnet 104 includes a static magnetic field generation source of a permanent magnet type, a resistive type, or a superconducting type. The gradient magnetic field coil 106 generates a gradient magnetic field in the imaging space. The gradient magnetic field coil 106 is composed of gradient magnetic field coils in directions of three axes including X, Y, and Z forming a stationary coordinate system that is a real space coordinate system. Each gradient magnetic field coil is connected to the gradient magnetic field power supply 116 and is supplied with a current. Accordingly, gradient magnetic fields are generated in the directions of three axes including X, Y, and Z.

The RF transmit coil 108 is a coil that irradiates the subject Exa with a radio-frequency magnetic field pulse. The radio-frequency magnetic field pulse may be referred to as an RF pulse. The RF transmit coil 108 is connected to the radio-frequency magnetic field generator 112 and is supplied with a radio-frequency pulse current. The radio-frequency magnetic field generator 112 is driven in accordance with an instruction from the sequencer 118 to modulate an amplitude of the radio-frequency pulse and supplies the amplified radio-frequency pulse to the RF transmit coil 108.

The sequencer 118 instructs the radio-frequency magnetic field generator 112 and the gradient magnetic field power supply 116 to generate a radio-frequency magnetic field and the gradient magnetic fields, respectively, in accordance with an imaging pulse sequence. The generated radio-frequency magnetic field is applied to the subject Exa as a pulsed radio-frequency magnetic field through the RF transmit coil 108. Accordingly, a nuclear magnetic resonance phenomenon is induced by spins of atoms constituting a biological tissue of the subject Exa. Nuclear magnetic resonance may be referred to as NMR using the abbreviation for Nuclear Magnetic Resonance.

The MRI device 100 comprises a receive coil unit. The receive coil unit is a coil that receives an echo signal released by the NMR phenomenon of the spins of the atoms constituting the biological tissue of the subject Exa. The echo signal may be referred to as an NMR signal.

FIG. 2 does not illustrate the receive coil unit. The receive coil unit may be of a blanket type applied to imaging of a chest part, an abdomen part, and the like. The receive coil unit to be applied may vary depending on the part to be examined. For example, receive coil units for imaging various parts such as a head part, a spine part, an abdomen part, a leg part, and an arm part can be used. One or a plurality of receive coil units may be used for imaging performed once. A plurality of receive coil units for imaging different parts may be used together. The receive coil unit may be simply referred to as a receive coil. The NMR signal generated from the subject Exa is received using the receive coil unit and detected using the receiver 114.

A nuclear magnetic resonance frequency used as a reference for detection in the receiver 114 is set by the sequencer 118. The nuclear magnetic resonance frequency may be referred to as a detection reference frequency. The sequencer 118 performs a control for operating each part by applying timings and intensity programmed in advance. In particular, a program that describes timings and intensity of the RF pulse, the gradient magnetic fields, and signal reception is referred to as a pulse sequence. Pulse sequences for various purposes are known.

The operation portion 140 includes a mouse, a keyboard, and the like and functions as a part of a GUI that receives input of an operator using a display operation window displayed on the display portion 142. That is, the operation portion 140 and the display portion 142 function as a GUI for causing the operator to start, stop, and pause the MRI device 100, select the pulse sequence, and input an imaging condition, a processing condition, and the like. GUI is the abbreviation for Graphical User Interface.

The controller 150 controls an operation of the MRI device 100 through the sequencer 118 and performs various types of signal processing such as image reconstruction by receiving the signal detected using the receiver 114.

The set detection reference frequency is applied to the receiver 114, and the receiver 114 performs quadrature detection of the echo signal that is an analog wave, converts the echo signal into raw data, and transmits the raw data to the controller 150. The raw data is referred to as the echo signal or measurement data.

The controller 150 acquires biological information of the subject Exa, and a biological information value calculated from the acquired biological information is set as an imaging parameter. The controller 150 executes a synchronized imaging task synchronized with a biological signal using the biological signal indicating the acquired biological information as a trigger.

The biological information detection device 152 comprises a sensor that detects the biological information of the subject Exa. The biological information detection device 152 may be an electrocardiograph that detects electrocardiogram information of the subject Exa. The biological information detection device 152 may be a respiratory function determination device that detects respiratory information of the subject Exa. The biological information detection device 152 may be a device separated from the MRI device 100.

The biological information value calculation portion 154 calculates the biological information value from the biological signal output from the biological information detection device 152. Examples of the biological information value include a heart rate and a respiratory rate. The controller 150 acquires the biological information value and sets the biological information value as the imaging parameter.

The controller 150 controls each part of the MRI device 100 in an integrated manner by receiving various instruction inputs from the operation portion 140 and generates an MRI image by performing processing such as converting the echo signal in a spatial frequency domain received through the sequencer 118 into a real-space image through inverse Fourier transform.

The controller 150 can be composed of a computer. The computer applied to the controller 150 may be a personal computer or a workstation. That is, the controller 150 comprises a processor and a memory, and the processor implements various functions of the MRI device 100 by executing a program including instructions stored in the memory.

A device comprising the controller 150 and the biological information value calculation portion 154 is an example of an imaging control device that sets an imaging parameter to be applied to imaging in the medical image capturing device. The imaging control device may comprise the biological information detection device 152.

### Procedure of Imaging Parameter Setting Method of Medical Image According to Embodiment

In an imaging parameter setting method of a medical image according to the embodiment, in performing examination including one or more electrocardiogram-synchronized imaging tasks, the heart rate or the like of the subject Exa is automatically set as a second set value for Beat Rate that is one of imaging parameters. The second set value of Beat Rate may be manually set by the operator.

In a case where Beat Rate is significantly changed from a first set value that is a default value set in advance, it is determined that there is an imaging parameter that needs to be changed because of the change in Beat Rate, and notification of options related to adjustment of the imaging parameter that needs to be changed because of the change in Beat Rate is provided.

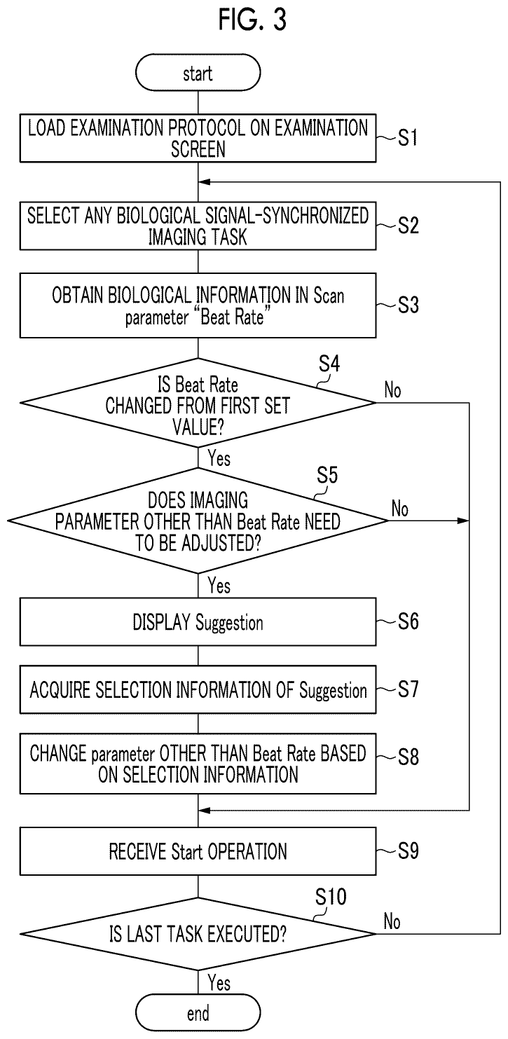

FIG. 3 is a flowchart illustrating a procedure of the imaging parameter setting method of the medical image according to the embodiment. In step S1, the controller 150 illustrated in FIG. 2 loads an examination protocol on an examination screen displayed on the display portion 142. That is, information related to the examination protocol is displayed on the examination screen.

In a case where the examination protocol is loaded, the first set value of the imaging parameter is set for each imaging task. The default value of the imaging parameter may be referred to as an initial value. The step of setting the default value of the imaging parameter may be executed separately from step S1.

In step S2, in a case where the operator selects any synchronized imaging task from synchronized imaging tasks displayed on the examination screen, the controller 150 acquires selection information of the synchronized imaging task.

In step S3, the controller 150 acquires the biological information value generated using the biological information value calculation portion 154. The controller 150 sets the acquired biological information value as the second set value for Beat Rate.

That is, in step S3, the second set value is set for Beat Rate at a timing at which any of the one or more synchronized imaging tasks displayed on the examination screen is selected. The timing at which the synchronized imaging task is selected may include a timing reached in any order of execution of the synchronized imaging tasks in a case where a plurality of synchronized imaging tasks are executed in order. Scan parameter in step S3 illustrated in FIG. 3 indicates the imaging parameter. Obtaining of the biological information value in step S3 is synonymous with acquisition of the second set value that is the biological information value, reception of the second set value that is the biological information value, and the like.

In step S4, the controller 150 determines whether or not the second set value set for Beat Rate in step S3 is changed from the first set value set in advance.

In step S4, in a case where the controller 150 determines that the second set value is not changed from the first set value, a No determination is made. In a case where a No determination is made, the procedure proceeds to step S9. Meanwhile, in step S4, in a case where the controller 150 determines that the second set value is changed from the first set value, a Yes determination is made. In a case where a Yes determination is made, the procedure proceeds to step S5.

In step S5, the controller 150 determines whether or not other imaging parameters other than Beat Rate need to be adjusted because of the change in Beat Rate. In step S5, in a case where the controller 150 determines that other imaging parameters other than Beat Rate do not need to be adjusted, a No determination is made. In a case where a No determination is made, the procedure proceeds to step S9.

Meanwhile, in step S5, in a case where the controller 150 determines that other imaging parameters other than Beat Rate need to be adjusted, a Yes determination is made. In a case where a Yes determination is made, the procedure proceeds to step S6.

In step S6, the controller 150 displays options of the imaging parameter that needs to be adjusted on the display portion 142. Suggestion in step S6 indicates suggestion of the options.

The controller 150 calculates and stores an adjusted value of the imaging parameter suggested to be adjusted in step S6 and adjusted values of imaging parameters different from Beat Rate. The controller 150 calculates and stores an adjusted value of the imaging parameter not suggested to be adjusted among imaging parameters that need to be adjusted because of the change in Beat Rate.

Beat Rate is an example of a first imaging parameter of the present disclosure. The imaging parameter suggested to be adjusted is an example of a second imaging parameter of the present disclosure. The imaging parameter not suggested to be adjusted among the imaging parameters that need to be adjusted because of the change in Beat Rate is an example of a third imaging parameter different from the second imaging parameter of the present disclosure.

In step S7, in a case where any of the options related to adjustment of the imaging parameter displayed on the display portion 142 is selected, the controller 150 acquires selection information related to adjustment of the imaging parameter.

In step S8, the controller 150 adjusts all imaging parameters that need to be adjusted because the change in Beat Rate, based on the selection information acquired in step S7. That is, the imaging parameter suggested to be adjusted and imaging parameters different from the imaging parameter suggested to be adjusted are automatically adjusted.

In step S9, the controller 150 receives an imaging start operation. In step S9, in a case where the imaging start operation is received, the controller 150 executes the selected imaging task.

In step S10, the controller 150 determines whether or not the last imaging task is executed each time the imaging task is finished. In step S10, in a case where the controller 150 determines that the last imaging task is not executed, a No determination is made. In a case where a No determination is made, the procedure proceeds to step S2, and each step from step S2 to step S10 is repeatedly executed until a Yes determination is made in step S10.

Meanwhile, in step S10, in a case where the controller 150 determines that the last imaging task is executed, a Yes determination is made. In a case where a Yes determination is made, determined finish processing is executed, and the procedure of the imaging parameter setting method of the medical image is finished.

In the imaging parameter setting method of the medical image according to the embodiment, step S4 and step S5 may be executed as one step. That is, a range of Beat Rate causing the imaging parameter that needs to be adjusted because of the change in Beat Rate may be determined in advance, and a step of determining whether or not the change in Beat Rate exceeds the determined range may be executed. In step S6, in a case where the change in Beat Rate exceeds the determined range, the options of the imaging parameter that needs to be adjusted may be displayed on the display portion 142.

In a case where the biological information value changes during execution of any imaging task, Beat Rate may be changed in the subsequent imaging task to be executed after the imaging task being executed, and the adjusted values of the imaging parameters different from Beat Rate may be calculated. The adjusted values of the imaging parameters different from Beat Rate may be calculated during execution of the imaging task in which the biological information value changes.

### Configuration Example of Display Screen

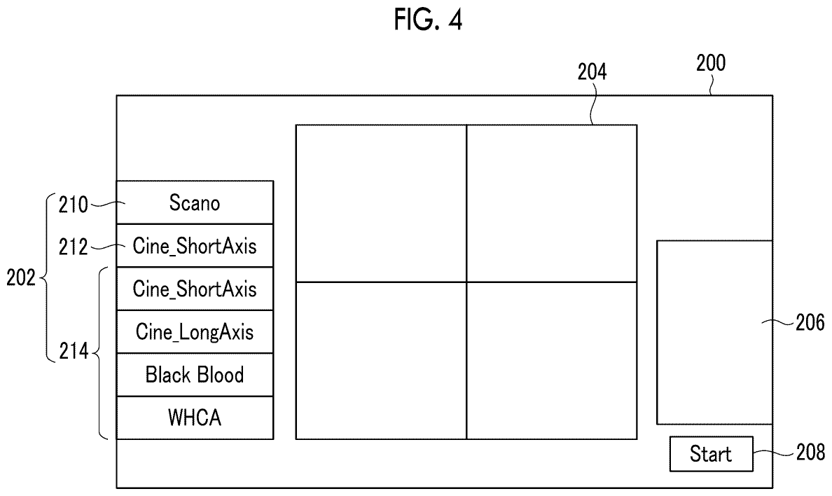

FIG. 4 is a schematic diagram illustrating a configuration example of a display screen. A display screen 200 illustrated in FIG. 4 is displayed on the display portion 142 illustrated in FIG. 2. A display provided in the gantry 110 may be applied to the display portion 142.

The display screen 200 displays a protocol display region 202, a positioning image display region 204, an imaging parameter setting region 206, an imaging start button 208, and the like. In the display screen 200, the protocol display region 202 is disposed in a left end portion, the positioning image display region 204 is disposed in a center portion, and the imaging parameter setting region 206 and the imaging start button 208 are disposed in a right end portion in FIG. 4.

The protocol display region 202 displays information related to the examination protocol used for imaging the subject Exa. The protocol display region 202 comprises a protocol identification information display region 210, a task selection information display region 212, and a task information display region 214.

The protocol identification information display region 210 displays identification information of the examination protocol such as a name of the loaded examination protocol. The task selection information display region 212 displays identification information of the imaging task such as a name of the selected imaging task among one or more imaging tasks included in the loaded examination protocol. The task selection information display region 212 may be displayed in a highlight manner.

FIG. 4 illustrates an aspect in which the identification information of the imaging tasks such as the names of one or more imaging tasks included in the loaded examination protocol is arranged in the task information display region 214 in an order of execution of the plurality of imaging tasks included in the examination protocol from top to bottom in FIG. 4.

The positioning image display region 204 displays a positioning image acquired by performing positioning imaging. FIG. 4 illustrates the positioning image display region 204 displaying four positioning images. Each region included in the positioning image display region 204 may display captured images to which cross sections different from each other are applied.

The imaging parameter setting region 206 displays the imaging parameter to be applied to imaging of the subject Exa. A pop-up type display aspect may be applied to the imaging parameter setting region 206.

The imaging start button 208 is a software button on which the operator performs an operation such as a click to start imaging. The controller 150 illustrated in FIG. 2 receives the operation of the imaging start button 208 and executes the selected imaging task.

### Specific Example of Imaging Parameter Setting Region

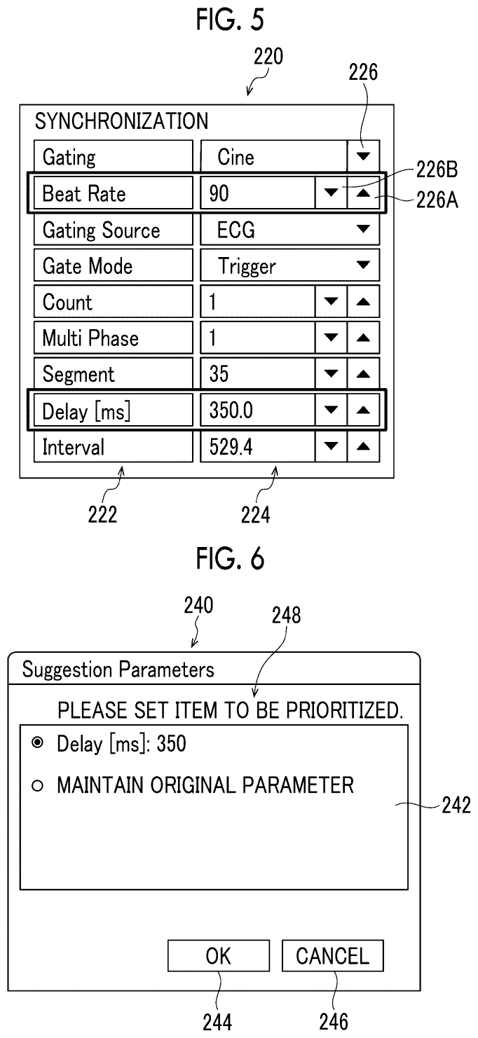

FIG. 5 is a schematic diagram illustrating a display example of the imaging parameter setting region illustrated in FIG. 4. FIG. 5 illustrates an imaging parameter display screen 220 displayed by causing the operator to click the imaging parameter setting region 206.

For example, in a case where the operator clicks the imaging parameter setting region 206, a pull-down type selection screen is displayed. The imaging parameter display screen 220 is displayed for the imaging parameter selected on the selection screen.

The imaging parameter display screen 220 illustrated in FIG. 5 includes an identification information display region 222 displaying identification information such as a name of each of a plurality of imaging parameters, and a setting display region 224 displaying settings for each imaging parameter.

Each part of the setting display region 224 illustrated in FIG. 5 displays an arrow tab 226 that is clicked to display a pull-down menu. The setting display region 224 of the imaging parameter for which a numerical value is set displays an upward arrow tab 226A for increasing the numerical value and a downward arrow tab 226B for decreasing the numerical value.

The first set value is set for each imaging parameter as a default value. The first set value of each imaging parameter may be the first set value obtained by adjusting a fixed value maintained in the MRI device 100 in accordance with a state and the like of the subject Exa. For example, in a case where 60 per minute is maintained for Beat Rate as a fixed value of the heart rate, and a measured value of the heart rate of the subject Exa is 65 per minute, 65 per minute may be set as the first set value of Beat Rate.

A delay period that is a period from a trigger timing to a timing at which main scanning starts is set as Delay. For example, 600.0 milliseconds may be set as the first set value corresponding to Beat Rate of 60 per minute.

FIG. 5 illustrates a case where Delay is adjusted to 350.0 milliseconds that is the second set value from the first set value because of the change in Beat Rate in a case where Beat Rate is changed to 90 per minute that is the second set value from the first set value.

FIG. 5 illustrates Gating, Beat Rate, Gating Source, Gate Mode, Count, Multi Phase, Segment, Delay, and Interval as the imaging parameters. Settings of imaging parameters related to each other may be linked to each other. For example, in a case where Cine indicating electrocardiogram-synchronized imaging is set as Gating, ECG indicating the electrocardiograph may be automatically set as Gating Source.

Specific Example of Parameter Adjustment Suggestion Information

### First Example

FIG. 6 is a schematic diagram illustrating a first example of a parameter adjustment suggestion information display screen. A parameter adjustment suggestion information display screen 240 illustrated in FIG. 6 is displayed on the imaging parameter display screen 220 illustrated in FIG. 5 in a case where Beat Rate is changed and there is an imaging parameter that needs to be adjusted because of the change in Beat Rate.

The parameter adjustment suggestion information display screen 240 illustrated in FIG. 6 includes a suggestion information display region 242, an OK button 244, a cancel button 246, and text information 248. The suggestion information display region 242 displays a plurality of suggestions related to adjustment of the imaging parameter.

FIG. 6 illustrates an example in which a suggestion to adjust Delay to 350 milliseconds and a suggestion to maintain the original setting without changing Delay are displayed as parameter suggestion information related to adjustment of the imaging parameter.

The OK button 244 is a button operated by the operator to confirm selection. The cancel button 246 is a button operated by the operator to cancel selection. FIG. 6 illustrates text information prompting the operator to make a selection as the text information 248.

FIG. 6 illustrates a state where the suggestion to adjust Delay to 350 milliseconds is selected. In a case where the operator operates the OK button 244, selection of the suggestion to adjust Delay to 350 milliseconds is confirmed. Meanwhile, in a case where the operator operates the cancel button 246, other options can be selected.

In a case where adjustment of the imaging parameter for adjusting Delay to 350 milliseconds is confirmed, the controller 150 illustrated in FIG. 2 changes set values of other imaging parameters that need to be adjusted because of adjustment of Delay to 350 milliseconds, to calculated adjusted values.

### Second Example

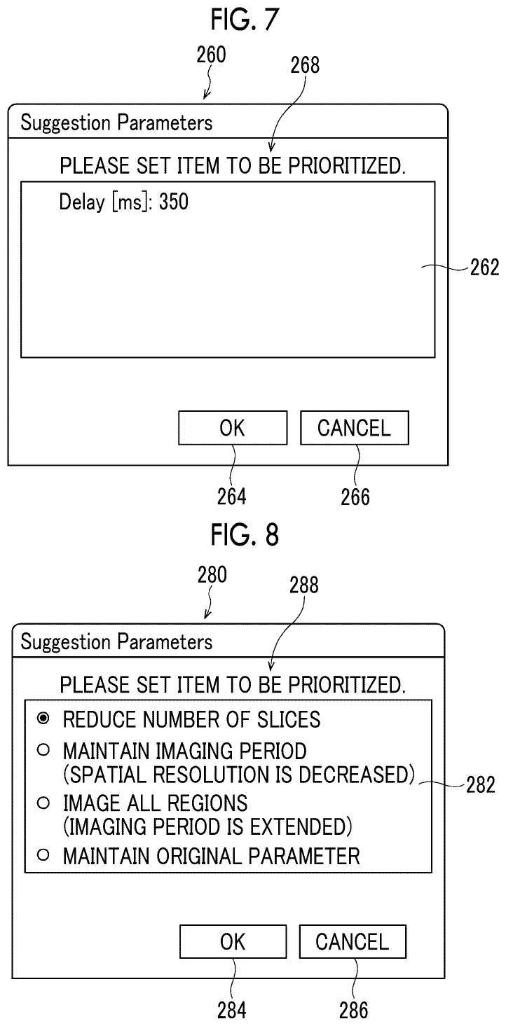

FIG. 7 is a schematic diagram illustrating a second example of the parameter adjustment suggestion information display screen. A parameter adjustment suggestion information display screen 260 illustrated in FIG. 7 includes a suggestion information display region 262, an OK button 264, a cancel button 266, and text information 268. Each of the OK button 264, the cancel button 266, and the text information 268 has the same function as the OK button 244 and the like illustrated in FIG. 6. The OK button 264 and the like will not be described.

While the suggestion information display region 242 illustrated in FIG. 6 illustrates two types of options, the suggestion information display region 262 illustrated in FIG. 7 displays only one type of option indicating the suggestion to adjust Delay to 350 milliseconds.

In selecting adjustment of the imaging parameter for adjusting Delay to 350 milliseconds, the operator operates the OK button 264 to confirm selection of adjustment of the imaging parameter for adjusting Delay to 350 milliseconds. Meanwhile, in maintaining the original setting without changing Delay, the operator operates the cancel button 266 to confirm maintaining of the original setting.

### Third Example

FIG. 8 is a schematic diagram illustrating a third example of the parameter adjustment suggestion information display screen. A parameter adjustment suggestion information display screen 280 illustrated in FIG. 8 includes a suggestion information display region 282, an OK button 284, a cancel button 286, and text information 288. Each of the OK button 284, the cancel button 286, and the text information 288 has the same function as the OK button 244 and the like illustrated in FIG. 6. The OK button 284 and the like will not be described.

While the suggestion information display region 242 illustrated in FIG. 6 specifically suggests the imaging parameter to be adjusted and the adjusted value, the suggestion information display region 282 illustrated in FIG. 8 displays options indicating effects on imaging. As the options, the suggestion information display region 282 displays a suggestion to reduce the number of slices, a suggestion to maintain an imaging period, a suggestion to image all regions, and a suggestion to maintain the original imaging parameter. The suggestion to maintain the imaging period indicates an effect on imaging such that spatial resolution is decreased. The suggestion to image all regions indicates an effect on imaging such that the imaging period is extended.

FIG. 8 illustrates a state where the suggestion to reduce the number of slices is selected. In a case where the operator operates the OK button 284, selection of the suggestion to reduce the number of slices is confirmed. Meanwhile, in a case where the operator operates the cancel button 286, other options can be selected.

In a case where adjustment of the imaging parameter for reducing the number of slices is confirmed, the controller 150 illustrated in FIG. 2 changes set values of other imaging parameters that need to be adjusted because of adjustment for reducing the number of slices, to calculated adjusted values.

### Specific Example of Adjustment of Imaging Parameter

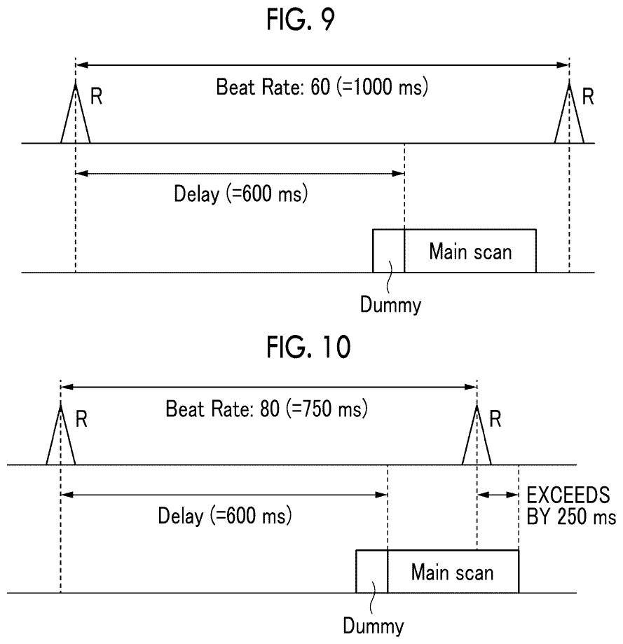

FIG. 9 is a timing chart illustrating an example of the imaging parameter in the electrocardiogram-synchronized imaging. FIG. 9 illustrates a timing chart in a case where 60 per minute is set as a set value of Beat Rate. Reference numeral R denotes an R wave of an electrocardiogram waveform functioning as a trigger for the synchronized imaging. In a case where the heart rate is 60 per minute, a period between any R wave and the subsequent R wave is 1000 milliseconds.

FIG. 9 illustrates a timing chart in a case where Trigger for performing imaging after an elapse of a certain period from the trigger is set as Gate Mode. In the example illustrated in FIG. 9, Delay indicating a time from the trigger to the main scanning is set to 600 milliseconds. The main scanning indicates capturing of the medical image used for diagnosis. Dummy illustrated in FIG. 9 indicates preliminary imaging performed before the main scanning.

An execution period of the main scanning is determined in accordance with the number of slices set as Segment. A set value of Delay and a set value of Segment are calculated in accordance with a setting of Beat Rate such that the period of Delay and the period of the main scanning fall in the period between any R wave and the subsequent R wave.

FIG. 10 is a timing chart illustrating a case where Beat Rate is changed. In a case where Beat Rate is changed to 80 per minute from 60 per minute, the period between any R wave and the subsequent R wave is changed to 750 ms from 1000 ms. In a case where the set value of Delay and the set value of Segment are maintained in the example illustrated in FIG. 9, a period required for one sequence exceeds the period between any R wave and the subsequent R wave by 250 milliseconds.

In such a case, the controller 150 illustrated in FIG. 2 determines that other imaging parameters other than Beat Rate need to be adjusted in step S5 illustrated in FIG. 5, and displays the options of the imaging parameter that needs to be adjusted, on the display portion 142 in step S6.

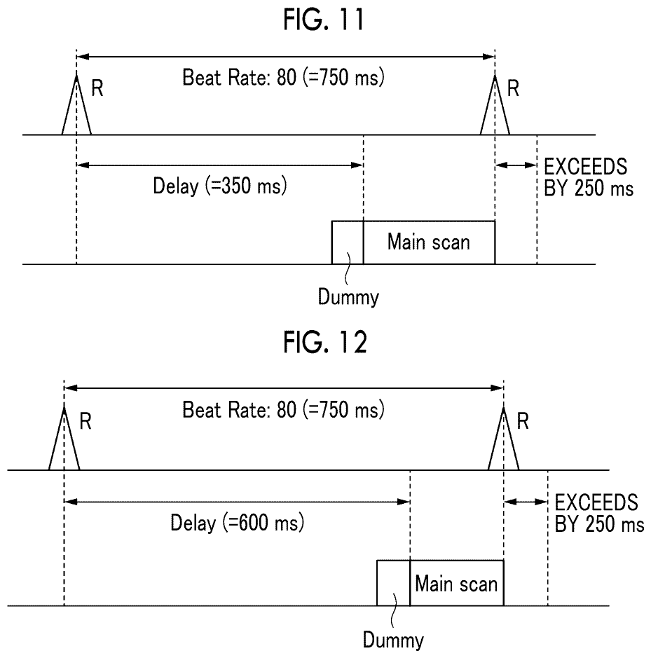

FIG. 11 is a schematic diagram illustrating a first change example of the imaging parameter. FIG. 11 illustrates an example in which Delay is adjusted to 350 milliseconds from 600 milliseconds as the imaging parameter that needs to be adjusted.

The controller 150 illustrated in FIG. 2 derives a suggestion to change the imaging parameter to avoid the period required for one sequence exceeding the period between any R wave and the subsequent R wave, by performing adjustment for reducing Delay by 250 milliseconds. The controller 150 derives the suggestion to maintain the original imaging parameter.

The controller 150 displays the suggestion to adjust the set value of Delay to 350 milliseconds from 600 milliseconds as one option in the suggestion information display region 242 on the parameter adjustment suggestion information display screen 240 illustrated in FIG. 6. The controller 150 displays the suggestion to maintain the original imaging parameter as one option in the suggestion information display region 242.

FIG. 12 is a schematic diagram illustrating a second change example of the imaging parameter. FIG. 12 illustrates an example in which the number of slices that is the set value of Segment is reduced as the imaging parameter that needs to be adjusted.

The controller 150 illustrated in FIG. 2 derives a suggestion to change the imaging parameter to avoid a case where the period required for one sequence exceeds the period between any R wave and the subsequent R wave, by reducing the number of slices that is the set value of Segment, to reduce the imaging period for performing the main scanning by 250 milliseconds to 350 milliseconds. The controller 150 derives the suggestion to maintain the original imaging parameter and displays the suggestion to maintain the original imaging parameter as one option in the suggestion information display region 242.

The controller 150 displays the suggestion to adjust the set value of Segment by reducing the number of slices as one option in the suggestion information display region 242 on the parameter adjustment suggestion information display screen 240 illustrated in FIG. 6.

### Another Display Example of Imaging Parameter Setting Region

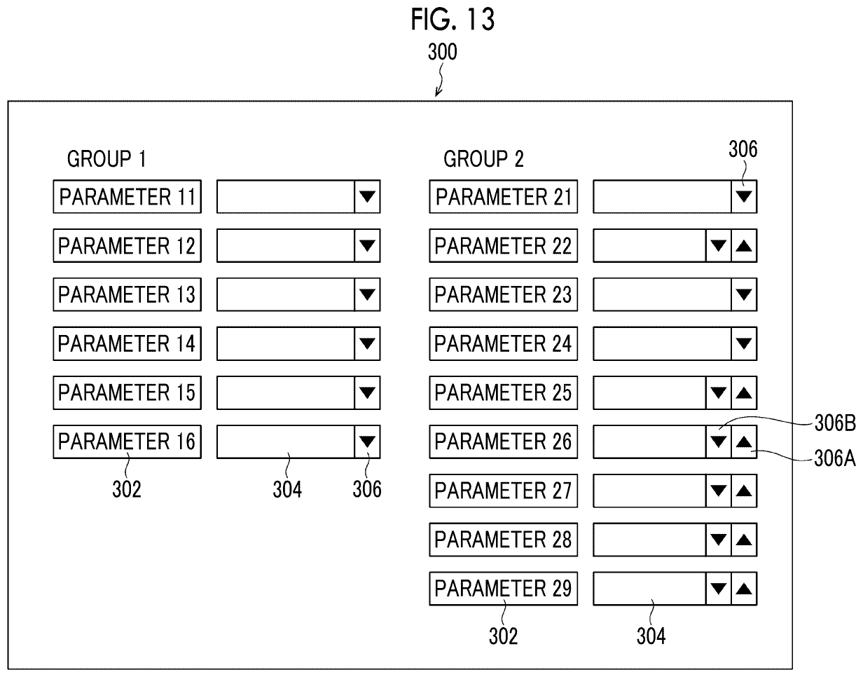

FIG. 13 is a schematic diagram illustrating another display example of the imaging parameter setting region. An imaging parameter setting screen 300 illustrated in FIG. 13 is displayed in the imaging parameter setting region 206 illustrated in FIG. 4.

In the imaging parameter setting screen 300, the plurality of imaging parameters are grouped for each function of the imaging parameters. FIG. 13 illustrates an example in which six imaging parameters including parameter 11 to parameter 16 are included as group 1, and nine imaging parameters including parameter 21 to parameter 29 are included as group 2.

While FIG. 13 illustrates two groups, the number of groups may be three or more. The number of each imaging parameter for each group is not limited to the aspect illustrated in FIG. 13 and may be appropriately determined. Disposition of the groups and disposition of each imaging parameter for each group are not limited to the example illustrated in FIG. 13 and may be appropriately determined.

The imaging parameter setting screen 300 displays an identification information display portion 302 displaying the identification information such as the names of the imaging parameters for each imaging parameter and a setting display portion 304 displaying settings for each imaging parameter.

Pop-up display is applied to the setting display portion 304. That is, the setting display portion 304 includes a pop-up display tab 306 operated by the operator to display a pop-up. The setting display portion 304 to which a numerical value is applied includes an increase tab 306A for displaying a pop-up for increasing the numerical value and a decrease tab 306B for displaying a pop-up for decreasing the numerical value.

In a case where the examination protocol for each subject Exa is loaded, a determined initial setting is set for each imaging parameter. The initial setting may be an initial setting maintained in the MRI device 100 or an initial setting determined for the loaded examination protocol. The initial setting may be a type of processing or a numerical value.

The operator may visually recognize the initial settings of the imaging parameters by displaying the imaging parameter setting screen 300. The operator may change the initial settings of the imaging parameters using the imaging parameter setting screen 300. The operator may, for example, change and adjust the imaging parameters for each task using the imaging parameter setting screen 300.

### Specific Example of Automatic Acquisition of Biological Information

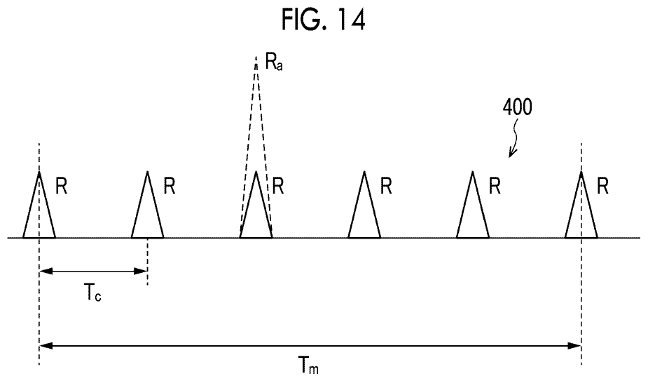

FIG. 14 is a schematic diagram of heart rate detection in calculating the heart rate. FIG. 14 schematically illustrates an electrocardiogram waveform 400 generated from an electrocardiogram signal output from the electrocardiograph. The electrocardiogram waveform 400 illustrated in FIG. 14 does not illustrate components other than the R wave, such as a P wave. Hereinafter, automatic acquisition of the heart rate will be illustrated as an example of automatic acquisition of the biological information. Reference numeral T_{c} denotes one cardiac cycle. Reference numeral Tₘ denotes a period for acquiring an output signal of the electrocardiograph applied in calculating the heart rate.

The biological information value calculation portion 154 illustrated in FIG. 2 calculates the heart rate based on the electrocardiogram signal acquired in a period of two or more cardiac cycles. FIG. 14 illustrates an example in which five cardiac cycles are applied to the period for acquiring the electrocardiogram signal.

For example, in a case where a sudden disturbance occurs in the heart rate of the subject Exa, and an electrocardiogram signal including an abnormal waveform Rₐ illustrated in FIG. 14 is acquired as the R wave, the measured value of the heart rate is temporarily rapidly increased. The temporarily rapidly increased heart rate is not preferable as the set value of Beat Rate.

In the MRI device 100, the heart rate applied to the synchronized imaging is calculated based on the electrocardiogram signal acquired in a certain period of two or more cycles in the electrocardiogram signal. Accordingly, the temporarily rapidly changed biological information value in a case where a disturbance in respiration caused by coughing occurs may be excluded from the set value of Beat Rate.

In a case where the respiratory rate is applied to the biological information value, the respiratory rate applied to the synchronized imaging is calculated based on a respiration signal acquired in a certain period of two or more cycles in the respiration signal, as in a case where the heart rate is applied.

### Abnormality Detection of Biological Information

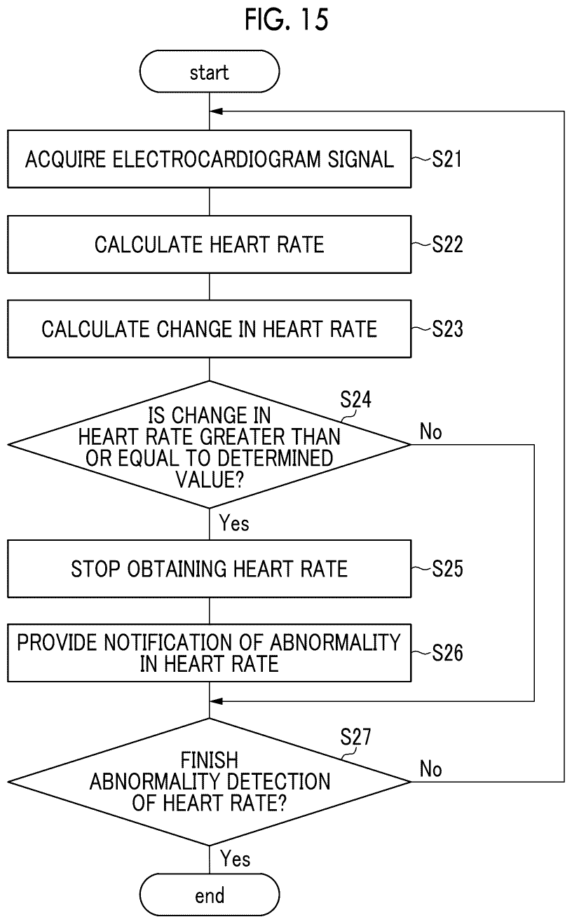

FIG. 15 is a flowchart illustrating a procedure of abnormality detection in the biological information. In a case where abnormality in the biological information is detected, the controller 150 illustrated in FIG. 2 provides notification of occurrence of the abnormality in the biological information without setting the biological signal value calculated from the biological information as the set value of Beat Rate.

In step S21, the biological information value calculation portion 154 illustrated in FIG. 2 acquires the electrocardiogram signal indicating the heart rate of the subject Exa from the electrocardiograph functioning as the biological information detection device 152. A determined sampling cycle is applied to acquisition of the electrocardiogram signal, and the electrocardiogram signal is consecutively acquired a plurality of times.

In step S22, the biological information value calculation portion 154 calculates the heart rate of the subject Exa from the electrocardiogram signal. The calculated heart rate may be stored in a time series order. Each time the heart rate is calculated, the latest heart rate may be stored, and the past heart rate other than the latest heart rate may be deleted.

In step S23, the biological information value calculation portion 154 calculates a change in the heart rate. Calculation of the change in the heart rate uses the electrocardiogram signals acquired at two consecutive sampling timings.

In step S24, the controller 150 determines whether or not the change in the heart rate is greater than or equal to a determined value. A reference value applied to the abnormality determination of the heart rate may be a fixed value. The reference value applied to the abnormality determination of the heart rate may be determined in accordance with an attribute and the like of the subject Exa.

In step S24, in a case where the controller 150 determines that the change in the heart rate is less than the determined value, a No determination is made. In a case where a No determination is made, the procedure proceeds to step S27. Meanwhile, in step S24, in a case where the controller 150 determines that the change in the heart rate is greater than or equal to the determined value, a Yes determination is made. In a case where a Yes determination is made, the procedure proceeds to step S25.

In step S25, the controller 150 stops obtaining the heart rate. In step S26, the controller 150 provides notification of the abnormality in the heart rate. That is, in step S26, notification of abnormality information indicating the abnormality in the heart rate is provided.

In step S27, the controller 150 determines whether or not to finish the abnormality detection of the heart rate. In a case where the abnormality detection of the heart rate continues, a No determination is made. In a case where a No determination is made, the procedure proceeds to step S21, and each step from step S21 to step S27 is repeatedly executed until a Yes determination is made in step S27.

Meanwhile, in a case where the abnormality detection of the heart rate is finished, a Yes determination is made. In a case where a Yes determination is made, determined finish processing is executed, and the abnormality detection of the heart rate is finished.

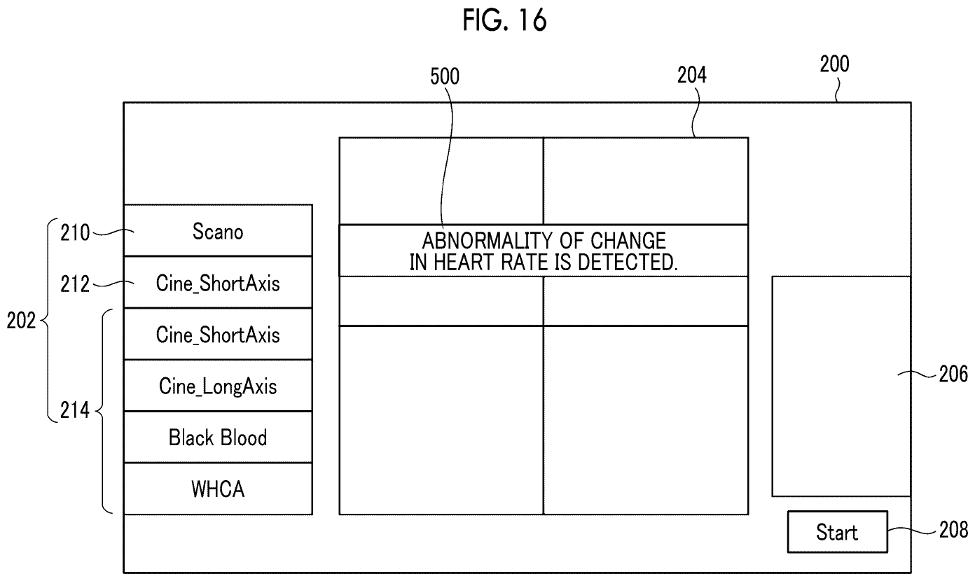

FIG. 16 is a schematic diagram illustrating a notification example of the abnormality in the biological information. Examples of the abnormality in the biological information include abnormality in attachment of the sensor that detects the biological information. FIG. 16 illustrates an example in which biological information abnormality detection information 500 is displayed in a superimposed manner on the display screen 200 illustrated in FIG. 4.

A display aspect of the biological information abnormality detection information 500 is not limited to the example illustrated in FIG. 16. For example, a dedicated screen on which the biological information abnormality detection information 500 is displayed may be displayed on the display portion 142 illustrated in FIG. 2.

Notification of the abnormality in the biological information is not limited to the aspect of using text information illustrated in FIG. 16. Notification of the abnormality in the biological information may use visual information such as blinking of a lamp for providing notification of the abnormality. Audio information such as a voice and an alarm sound may be applied to notification of the abnormality in the biological information.

### Effects of Embodiment

The MRI device 100 and the imaging parameter setting method according to the embodiment can achieve the following effects.
[1]

In the synchronized imaging to which the biological information is applied, the biological information value such as the heart rate and the respiratory rate is automatically set as the second set value of Beat Rate. Accordingly, occurrence of an erroneous setting and the like of Beat Rate is suppressed.
[2]

Other imaging parameters other than Beat Rate that need to be adjusted because of a change in the set value of Beat Rate are calculated. Accordingly, an operation of the operator related to the imaging parameters is simplified. Effects on a captured image that depends on a skill level of the operator are reduced.
[3]

A direction of adjustment is suggested for other imaging parameters other than Beat Rate that need to be adjusted because of a change in the set value of the Beat Rate. Accordingly, in a case where the set value of Beat Rate is changed, the operator can easily adjust other imaging parameters other than Beat Rate.
[4]

As a suggestion related to the direction of adjustment of the imaging parameters, effects and the like on the captured image obtained by performing the main scanning are displayed. Accordingly, the operator may adjust the imaging parameters by taking the effects on the captured image into consideration.
[5]

In a case where one examination protocol includes a plurality of synchronized imaging tasks, Beat Rate based on the latest biological information at a selection timing for each synchronized imaging task is set.
[6]

A value calculated from a certain period is applied to the biological information value set as Beat Rate. Accordingly, a case where sudden changes in the biological information such as the heart rate and respiration affects the set value of Beat Rate is avoided.
[7]

In a case where abnormality in the biological information such as the heart rate and respiration is detected, notification of the abnormality in the biological information is provided without setting the biological information value for Beat Rate. Accordingly, a case where the abnormality and the like in attachment of the sensor that detects the biological information affect the set value of Beat Rate is avoided.

The present disclosure is not limited to the above embodiment, and various modifications can be made without departing from the gist of the technical idea of the disclosed technology.

### EXPLANATION OF REFERENCES

100: MRI device
142: display portion
150: controller
152: biological information detection device
154: biological information value calculation portion

What is claimed is:

1. An imaging control device that sets an imaging parameter applied to imaging of a subject including a synchronized imaging task in which biological information of the subject is used, in a medical image capturing device which performs the imaging, the imaging control device comprising:
a processor; and
a memory storing a program to be executed by the processor,
wherein the processor is configured to:
acquire the biological information of the subject;
in a case where a second set value based on the acquired biological information of the subject is changed from a first set value set in advance for a first imaging parameter for which the biological information of the subject is set, determine whether or not a second imaging parameter different from the first imaging parameter needs to be adjusted; and
in a case where a determination that the second imaging parameter needs to be adjusted is made, display parameter adjustment suggestion information related to adjustment of the second imaging parameter.

2. The imaging control device according to claim 1,
wherein the processor is configured to display a suggestion to adjust some of a plurality of the second imaging parameters as the parameter adjustment suggestion information.

3. The imaging control device according to claim 2,
wherein the processor is configured to:
acquire selection information for selecting the displayed suggestion to adjust the second imaging parameter; and
automatically adjust a third imaging parameter different from the selected second imaging parameter.

4. The imaging control device according to claim 1,
wherein the processor is configured to display information indicating an effect on the imaging of the subject as the parameter adjustment suggestion information.

5. The imaging control device according to claim 1,
wherein the processor is configured to automatically acquire the second set value.

6. The imaging control device according to claim 1,
wherein the processor is configured to:
in a case where a plurality of the synchronized imaging tasks are performed, acquire the biological information for each synchronized imaging task; and
set a latest second set value for each synchronized imaging task.

7. The imaging control device according to claim 1,
wherein the processor is configured to calculate the second set value based on the biological information acquired in a period of two or more cycles of a repeating cycle of the biological information.

8. The imaging control device according to claim 1,
wherein the processor is configured to:
acquire a change in a biological information value that is a value of the biological information; and
in a case where the acquired change in the biological information value exceeds a determined value, provide notification of abnormality information indicating abnormality in the biological information value.

9. The imaging control device according to claim 1,
wherein the processor is configured to, in a case where the determination that the second imaging parameter needs to be adjusted is made, display a plurality of pieces of the parameter adjustment suggestion information.

10. The imaging control device according to claim 1,
wherein the processor is configured to determine whether or not the second set value based on the acquired biological information of the subject is changed from the first set value set in advance for the first imaging parameter for which the biological information of the subject is set.

11. The imaging control device according to claim 1,
wherein the processor is configured to, in a case where a change in the second set value from the first set value of the first imaging parameter is greater than or equal to a determined value, determine that the second imaging parameter needs to be adjusted.

12. A medical image capturing device that performs imaging of a subject including a synchronized imaging task in which biological information of the subject is used, the medical image capturing device comprising:
a processor; and
a memory storing a program to be executed by the processor,
wherein the processor is configured to:
acquire the biological information of the subject;
in a case where a second set value based on the acquired biological information of the subject is changed from a first set value set in advance for a first imaging parameter for which the biological information of the subject is set, determine whether or not a second imaging parameter different from the first imaging parameter needs to be adjusted; and
in a case where a determination that the second imaging parameter needs to be adjusted is made, display parameter adjustment suggestion information related to adjustment of the second imaging parameter.

13. An imaging parameter setting method performed by a computer functioning as a medical image capturing device that performs imaging of a subject including a synchronized imaging task in which biological information of the subject is used, the imaging parameter setting method comprising:
acquiring the biological information of the subject;
determining, in a case where a second set value based on the acquired biological information of the subject is changed from a first set value set in advance for a first imaging parameter for which the biological information of the subject is set, whether or not a second imaging parameter different from the first imaging parameter needs to be adjusted; and
displaying, in a case where a determination that the second imaging parameter needs to be adjusted is made, parameter adjustment suggestion information related to adjustment of the second imaging parameter.

14. A non-transitory, computer readable tangible recording medium which records thereon, a program for causing a computer functioning as a medical image capturing device that performs imaging of a subject including a synchronized imaging task in which biological information of the subject is used, to implement:
a function of acquiring the biological information of the subject;
a function of determining, in a case where a second set value based on the acquired biological information of the subject is changed from a first set value set in advance for a first imaging parameter for which the biological information of the subject is set, whether or not a second imaging parameter different from the first imaging parameter needs to be adjusted; and
a function of displaying, in a case where a determination that the second imaging parameter needs to be adjusted is made, parameter adjustment suggestion information related to adjustment of the second imaging parameter.
